(19) United States
(12) Patent Application Publication
(10) Pub. No.: US 2025/0268928 A1
(43) Pub. Date: Aug. 28, 2025

(54) USE OF BACTERIORUBERINS AND GLYCOSYL DERIVATIVES THEREOF TO PREVENT AND TREAT DISEASES INVOLVING DYSREGULATED PROTEIN AGGREGATION, SUCH AS NEURODEGENERATIVE DISEASES

(71) Applicants: Jean-Noel THOREL, Paris (FR); Miroslav RADMAN, Le Kremlin Bicetre (FR); NAOS INSTITUTE OF LIFE SCIENCE, Aix En Provence (FR)

(72) Inventors: Jean-Noel THOREL, Paris (FR); Miroslav RADMAN, Le Kremlin Bicetre (FR)

(21) Appl. No.: 18/257,440

(22) PCT Filed: Dec. 16, 2021
(86) PCT No.: PCT/EP2021/086302

§ 371 Date: Jun. 14, 2023

(30) Foreign Application Priority Data
Dec. 16, 2020 (FR) 2013390

(51) Int. Cl. A61K 31/7032; A61K 31/045; A61K 35/74; A61P 25/28

(52) U.S. Cl. CPC A61K 31/7032; A61K 31/045; A61K 35/74; A61P 25/28

(57) ABSTRACT

The invention concerns a composition comprising at least one bacterioruberin, preferably in the glycosylated form, optionally as a mixture with different forms of glycosylated bacterioruberins, or an extract comprising it, for its use in a method for the treatment or prevention of a disease involving a deregulation in protein aggregation such as a degenerative disease, for example, advantageously a neurodegenerative disease, fibrosis, advantageously pulmonary fibrosis, or diabetes. The invention also concerns a bacterioruberin, preferably in the glycosylated form, optionally as a mixture with different forms of glycosylated bacterioruberins, or an extract comprising it, for its use in a method for the treatment or prevention of a disease involving a deregulation in protein aggregation, and a method for the treatment or prevention of a degenerative disease.

### CROSS-REFERENCE TO RELATED APPLICATIONS

This application is the U.S. National Phase under 35 U.S.C. § 371 of International Application PCT/EP2021/086302, filed Dec. 16, 2021, which claims the benefit of French Application No. 2013390, filed on Dec. 16, 2020, the disclosures of which are incorporated by reference herein in their entireties.

### FIELD

The present invention relates to a composition comprising at least one bacterioruberin and/or at least one glycosylated bacterioruberin for the treatment or prevention of a disease involving a deregulation of protein aggregation, such as degenerative diseases, advantageously neurodegenerative diseases, in particular a disease selected from Alzheimer's disease (AD), Parkinson's disease (PD), Huntington's disease, posterior cortical atrophy or in fact amyotrophic lateral sclerosis (ALS), as well as ocular neurodegenerative diseases selected from macular degeneration, retinitis pigmentosa and retinopathy.

### PRIOR ART

Degenerative diseases, and in particular neurodegenerative diseases such as Alzheimer's disease (AD), Parkinson's disease (PD), Huntington's disease, posterior cortical atrophy or in fact amyotrophic lateral sclerosis, as well as ocular neurodegenerative diseases, are slowly progressive, disabling chronic diseases. They usually cause a deterioration in the function of nerve cells, in particular neurons, which can lead to cell death or neurodegeneration. The disorders induced by neurodegenerative diseases are varied and may be of the cognitive-behavioural, sensory and motor type (Dugger et al. 2017).

It is difficult to gauge the overall impact of neurodegenerative diseases on the global human population; the World Health Organization (WHO) estimates that up to one billion human beings could be affected if all the manifestations of these conditions are considered, the limits of which are sometimes unclear; these figures are likely to increase in view of the increasing ageing of the population in developed and developing countries.

As research progresses, many similarities appear which link these diseases to each other, especially on the cellular level through the aggregation of atypical or unfolded proteins and induced neuronal death. The discovery of these similarities offers hope for therapeutic advances that could simultaneously improve many diseases, in particular by acting on the mechanisms of intracellular protein aggregation in neurons.

Carotenoids are highly conjugated linear isoprenoid compounds which are responsible for the majority of the yellow, orange and red pigmentation observed in organisms on Earth (Armstrong, 1997). Carotenoid biosynthesis occurs in all living things, with the exception of animals in which carotenoids are introduced through diet (Britton, 1995). Although about a thousand different carotenoids have been identified in nature and they have very varied structural characteristics, all known carotenoids share a conjugated linear lipophilic backbone, obtained by way of highly conserved biosynthetic pathways (Britton, 2004). Carotenoids are synthesized by the linear condensation of isoprene units derived from primary metabolism (Armstrong, 1994). Covalent modifications at each end of the chain give rise to the observed structural diversity of known carotenoids (Armstrong 1997). Desaturation of the chains generates the chromophore which is characteristic of carotenoids, which results in a region of readily excitable delocalized electrons; these properties are the basis of two fundamental characteristics which are common to all carotenoids, namely their photochemical properties and their antioxidant action (Britton, 1995).

The term “carotenoid” includes the molecules of the carotene and xanthophyll families.

Among the carotenoids with the greatest antioxidant potential, mention should be made of bacterioruberins, which are tetrahydroxylated carotenes containing 50 carbon atoms. Bacterioruberins and their derivatives are found in extremophile bacteria, in particular halophilic archaea and certain psychrophilic actinobacteria; in these microorganisms, they play an important role in the protection of the DNA and the membranes against solar radiation as well as the thermal and osmotic environmental stresses to which these organisms are permanently exposed (Mandelli et al. 2012). In particular, these carotenes are found in the psychrophilic actinobacterium Arthrobacter (Micrococcus) agilis. This bacterium is also capable of synthesizing glycosylated forms of bacterioruberins, i.e., in which the terminal hydroxyl groups are substituted with sugars (Fong et al. 2001).

Several carotenoids are known which can help prevent, slow down or treat neurodegenerative diseases. Thus, patent application WO 2014/155189 discloses the use of several xanthophylls, in particular lutein and zeaxanthin, for the treatment and prevention of PD and AD.

Application WO 2008/038119 discloses the treatment of PD with a composition containing: (a) a complex of coenzyme Q10 and at least one cyclodextrin; and (b) at least one carotenoid, in particular a carotene selected from α-carotene, β-carotene and lycopene.

It is also known that glycosylated carotenoids may be useful in the treatment and prevention of neurodegenerative diseases: by way of example, a neuroprotective action has been associated with crocin, which is a glycosylated carotenoid responsible for the yellow colour of saffron (Farkhondeh et al. 2018).

Despite the usefulness of the carotenoids already employed in the prevention of neurodegenerative diseases, there remains a clear need for novel remedies which are capable of combatting the emergence of these pathologies more effectively.

### Aims of the Invention

The aim of the invention is to solve the technical problem consisting of providing a compound or a composition having a chaperone activity, i.e., having the ability to combat protein denaturing and aggregation, thereby protecting cellular proteins.

Thus, the invention also aims to solve the technical problem consisting of providing a compound or a composition protecting at least one intracellular or extracellular protein from both oxidative stress and denaturing.

It is intended to solve the technical problem consisting of providing a compound or composition which is useful in the treatment and prevention of a disease such as degenerative diseases, for example, exhibiting a deregulation in protein aggregation.

### DESCRIPTION OF THE INVENTION

Surprisingly, the Applicant has discovered that a bacterioruberin, preferably in the glycosylated form, optionally as a mixture with various forms of glycosylated bacterioruberins, or an extract comprising them, has a chaperone activity, thus making them useful in the treatment and prevention of diseases such as degenerative diseases, for example, involving a deregulation in protein aggregation, advantageously a neurodegenerative disease, fibrosis, advantageously pulmonary fibrosis, or diabetes. Examples of neurodegenerative diseases which may in particular be cited are neurodegenerative diseases characterized by the accumulation of protein aggregates in neurons, such as AD and PD. The experimental section demonstrates that the bacterioruberins, and moreover the glycosylated bacterioruberins, make it possible to stabilize the proteins and slow down their inactivation/denaturing. The chaperone effect of these molecules can play an important role in the treatment of these pathologies, by protecting neurons.

The present invention also concerns a composition comprising a bacterioruberin, preferably in the glycosylated form, optionally mixed with different forms of glycosylated bacterioruberins, or an extract comprising it, for its use in the treatment or prevention of a disease involving a deregulation in protein aggregation such as a degenerative disease, for example, advantageously a neurodegenerative disease, fibrosis, advantageously pulmonary fibrosis, or diabetes.

In particular, the invention concerns a composition comprising a bacterioruberin, preferably in the glycosylated form, optionally as a mixture with different forms of glycosylated bacterioruberins, or an extract comprising it, for its use in the treatment or prevention of a disease involving a deregulation in protein aggregation by reducing the formation of toxic protein aggregates, and in particular in neurons.

In particular, the invention concerns a composition comprising a bacterioruberin, preferably in the glycosylated form, optionally as a mixture with different forms of glycosylated bacterioruberins, or an extract comprising it, for its use in the treatment or prevention of a disease involving a deregulation in protein aggregation by reducing protein denaturing.

Thus, the present invention concerns a bacterioruberin, preferably in the glycosylated form, optionally as a mixture with different forms of glycosylated bacterioruberins, or an extract comprising it, for its use in the treatment or prevention of a degenerative disease, advantageously a neurodegenerative disease.

The present invention also concerns a composition comprising a bacterioruberin, preferably in the glycosylated form, optionally as a mixture with different forms of glycosylated bacterioruberins, or an extract comprising it, for its use in the treatment or prevention of a degenerative disease, advantageously a neurodegenerative disease.

The present invention also concerns a method for treating or preventing a degenerative disease, advantageously a neurodegenerative disease, in which a composition comprising at least one bacterioruberin and/or at least one glycosylated bacterioruberin is administered to a subject in need thereof.

Typically, the neurodegenerative disease is selected from Alzheimer's disease (AD), Parkinson's disease (PD), Huntington's disease, posterior cortical atrophy or in fact amyotrophic lateral sclerosis (ALS) as well as ocular neurodegenerative diseases selected from macular degeneration, retinitis pigmentosa and retinopathy, advantageously for the treatment of Alzheimer's disease (AD) or Parkinson's disease (PD).

The compositions in accordance with the invention may also be useful for the treatment of other pathologies involving a deregulation in protein aggregation such as, for example, fibrosis, advantageously pulmonary fibrosis, or diabetes.

Bacterioruberin (CAS No 32719-43-0), is also known as “α-bacterioruberin”.

“α-bacterioruberin” has the following structure:

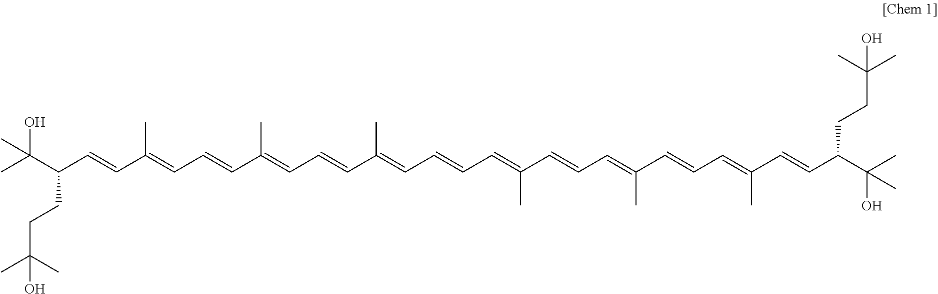 [SMILES: CC(/C=C/C=C(C)/C=C/C=C(C)/C=C/[C@H](CCC(C)(C)O)C(C)(C)O)=C\C=C\C=C(C)\C=C\C=C(C)\C=C\C=C(C)\C=C\[C@H](CCC(C)(C)O)C(C)(C)O]

α-bacterioruberin comprises 4 terminal hydroxyl groups, each of which being capable of being substituted by ether bonding with a sugar-type group, or even one or more covalently bonded sugars. The term “glycosylated form of bacterioruberin” or “glycosylated bacterioruberin” means a bacterioruberin in which at least one hydroxyl group is substituted with one or more, for example two or three, sugar residues by means of an ether bond between the backbone of the bacterioruberin and the sugar.

An “isolated glycosylated bacterioruberin” in accordance with the invention is obtained by biotechnological synthesis, by chemical synthesis, typically followed by purification, or alternatively by purification of a glycosylated bacterioruberin naturally contained in a natural bacterium.

As an example, a “glycosylated bacterioruberin” in accordance with the invention corresponds to the following structure:

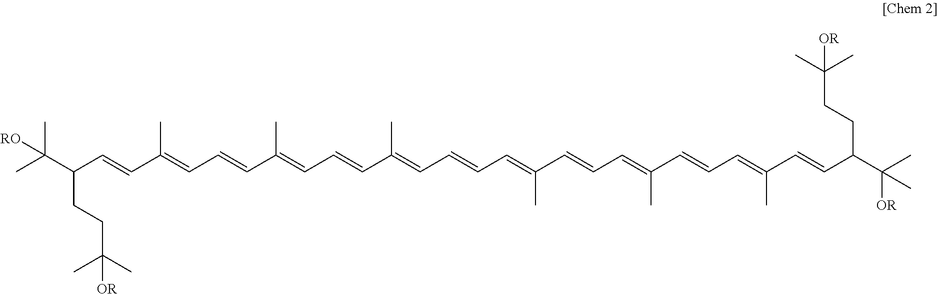

in which R is independently selected from a hydrogen atom, one or more, for example two or even three, sugar residues, and wherein in at least one occurrence, R represents one or more, for example two or even three, sugar residues.

In a preferred embodiment, the sugar is a hexose or a deoxyhexose selected from the group constituted by allose, altrose, glucose, mannose, gulose, idose, galactose, fucose, fructose and fucose.

In a preferred implementation, a composition in accordance with the invention comprises at least one glycosylated bacterioruberin selected from monoglycosylated bacterioruberins, diglycosylated bacterioruberins, triglycosylated bacterioruberins, tetraglycosylated bacterioruberins, pentaglycosylated bacterioruberins, hexaglycosylated bacterioruberins, heptaglycosylated bacterioruberins, octaglycosylated bacterioruberins, nonaglycosylated bacterioruberins, decaglycosylated bacterioruberins, undecaglycosylated bacterioruberins, and dodecaglycosylated bacterioruberins. Advantageously, it is at least one glycosylated bacterioruberin selected from monoglycosylated bacterioruberins, diglycosylated bacterioruberins, triglycosylated bacterioruberins and tetraglycosylated bacterioruberins.

Advantageously, said composition comprises a mixture of monoglycosylated bacterioruberins, diglycosylated bacterioruberins and tetraglycosylated bacterioruberins; and preferably a mixture of monoglycosylated bacterioruberins and diglycosylated bacterioruberins. In a preferred embodiment, the composition of the invention is essentially free from non-glycosylated forms of bacterioruberin.

Advantageously, the total extract of carotenoids containing the glycosylated bacterioruberins in accordance with the invention is a bacterial extract, preferably from Actinobacteria, yet more advantageously from the Microccoccaceae family. Advantageously, the species are Micrococcus roseus and Arthobacter agilis.

The glycosylated bacterioruberins may be obtained by extraction and purification, for example by chromatography, of total extracts of carotenoids from actinobacteria of the Micrococcus or Arthrobacter genera, advantageously the species A. agilis and/or M. roseus. The species A. agilis is also known as Micrococcus agilis. Thus, the extracts and strains described in the publications by Strand et al. 1997, Fong et al. 2001 and in patent application WO 2014/167247 may be used as a source of glycosylated bacterioruberins. Preferably, the strains of A. agilis used as sources of glycosylated bacterioruberins within the meaning of the invention are the strain MB813 (described in Fong et al. 2001) and/or SB5 (described in patent application WO 2014/167247). The methods for obtaining the extracts of total carotenoids from these bacterial species are known to the person skilled in the art and have been described, for example, in Strand et al. 1997, Fong et al. 2001 as well as patent application WO 2014/167247. However, these methods do not make it possible to isolate the various glycosylated bacterioruberins.

Surprisingly, the Applicant has developed a method which makes it possible to efficiently isolate the glycosylated forms of bacterioruberin from an extract of carotenoids of A. agilis. The present invention describes a method for purifying and isolating bacterioruberin and its glycosylated forms.

Thus, the present invention also concerns the isolated bacterioruberin and/or an isolated glycosylated bacterioruberin, as well as their mixtures, in particular for the uses and applications described in the present invention.

In other words, the invention encompasses a separated and purified glycosylated bacterioruberin, in particular from an extract of an extremophile bacterium, and preferably Arthrobacter agilis, for its use in a method for treating or preventing a degenerative disease, advantageously a neurodegenerative disease.

In a preferred embodiment, a total extract of carotenoids containing the glycosylated bacterioruberins in accordance with the invention corresponds to the carotenoids contained in the starting material MIRORUBERINE marketed by GREENTECH and corresponding to the INCI name Micrococcus lysate. Alternatively, the glycosylated bacterioruberins in accordance with the invention may be obtained by biotechnology, or by chemical synthesis, for example by means of a controlled glycosylation of the native forms of bacterioruberins, typically α-bacterioruberins, for example starting from the α-bacterioruberin. This glycosylation may be obtained chemically or by biotechnology, preferably by biotechnology using suitable glycosyltransferases. By way of example, the starting material HALORUBINE marketed by HALOTEK GmbH may be used as a source of α-bacterioruberin in the synthesis of glycosylated bacterioruberins within the meaning of the invention.

Advantageously, a composition in accordance with the invention comprises α-bacterioruberin. The α-bacterioruberin may be obtained by extracting the aforementioned actinobacteria, which also synthesize glycosylated forms of the bacterioruberin. Alternatively, the α-bacterioruberin may be extracted from cultures of one or more haloarchaea such as, for example, the species Halobacterium salinarum, Halorubrum sodomense, Haloarcula valismortis and Salinibacter ruber. Thus, the starting material HALORUBINE marketed by HALOTEK and corresponding to the INCI name Halobacterium salinarum carotenoides may be used in the compositions in accordance with the invention.

In an alternative embodiment, a composition in accordance with the invention comprises at least one bacterioruberin and a glycosylated bacterioruberin. In other words, this composition comprises a mixture of glycosylated forms of bacterioruberin and of non-glycosylated d forms, and advantageously a mixture of α-bacterioruberin, monoglycosylated bacterioruberins and diglycosylated bacterioruberins. Advantageously, the ratio between the non-glycosylated forms and the glycosylated forms is comprised between 2/1 and 1/2.

In one embodiment, a composition in accordance with the invention comprises one or more glycosylated bacterioruberins and essentially does not comprise a non-glycosylated form of bacterioruberin. The term “essentially does not comprise a non-glycosylated form of bacterioruberin” or “essentially free from non-glycosylated forms of bacterioruberin” means that although it is sought to avoid and eliminate the non-glycosylated form of bacterioruberin, it might be present in trace amounts. Preferably, such traces are not detectable by analysis.

Advantageously, a composition in accordance with the invention comprises a mixture of monoglycosylated bacterioruberins, diglycosylated bacterioruberins and tetraglycosylated bacterioruberins; and preferably a mixture of glycosylated bacterioruberins essentially constituted by monoglycosylated bacterioruberins and diglycosylated bacterioruberins, and said mixture preferably comprising 20 to 80% by weight of monoglycosylated bacterioruberins and 20 to 80% by weight of diglycosylated bacterioruberins with respect to the total weight of the mixture of glycosylated bacterioruberins.

The pharmaceutical compositions comprising at least one bacterioruberin and/or a glycosylated bacterioruberin in accordance with the invention are generally in dosage form. Thus, the composition comprising at least one bacterioruberin and/or a glycosylated bacterioruberin may be in the form of a tablet, a sugar-coated tablet, a capsule, a suppository, an injectable or oral solution, or in fact a drop, and it is capable of being administered orally, oromucosally, rectally, vaginally, parenterally intramuscularly, or ophthalmically.

Among the pharmaceutical compositions in accordance with the invention, more particular mention will be made of those which are suitable for oral, oromucosal, parenteral (intravenous, intramuscular or subcutaneous), per- or transcutaneous, intravaginal, rectal, nasal, perlingual, buccal, ocular or respiratory administration.

The pharmaceutical compositions in accordance with the invention for parenteral injections in particular include aqueous and non-aqueous sterile solutions, dispersions, suspensions or emulsions as well as sterile powders for reconstituting injectable solutions or dispersions.

For solid oral administration, the pharmaceutical compositions in accordance with the invention in particular include simple or sugar-coated tablets, sublingual tablets, sachets, capsules or granules, and for oral, nasal, buccal or ocular liquid administrations, in particular include emulsions, solutions, suspensions, drops, syrups and aerosols.

The pharmaceutical compositions for rectal or vaginal administration are preferably suppositories or ovules, and those for per- or transcutaneous administration in particular include powders, aerosols, creams, ointments, gels and patches.

The aforementioned pharmaceutical compositions illustrate the invention but do not limit it in any way.

Examples of excipients or vehicles which are inert, non-toxic, acceptable to a human being or pharmaceutically acceptable that may be cited by way of indication and without implying any limitation are diluents, solvents, preservatives, wetting agents, emulsifiers, dispersing agents, binders, blowing agents, disintegrating agents, retardants, lubricants, absorbents, suspending agents, dyes, flavourings, etc.

The useful dosage varies as a function of the age and weight of the patient, the mode of administration, the pharmaceutical composition used, the nature and the severity of the condition. By way of example, the composition in accordance with the invention may be administered once a month, once a week or daily and it may contain from 1 mg to 1 g of glycosylated bacterioruberins and/or non-glycosylated bacterioruberins or any of their mixtures.

The glycosylated bacterioruberins in accordance with the invention are suitable for their use in food and nutraceutical supplements. Methods for formulating food supplements are known to the person skilled in the art. Advantageously, the food supplements are in the form of a tablet or capsule. By way of example, each dose may contain from 1 mg to 1 g of glycosylated bacterioruberins and/or non-glycosylated bacterioruberins, and any of their mixtures.

In a tablet, microcrystalline cellulose is used as a bulking agent, for example. It is used in an amount of 10 to 30% by weight with respect to the total weight of the food supplement, more advantageously approximately 20% by weight.

Dicalcium phosphate and tricalcium phosphate are used as compression agents for the preparation of tablets. 10% to 30% by weight of dicalcium phosphate with respect to the total weight of the food supplement is used, more advantageously approximately 15% by weight. A quantity of 2.5% to 7.5% by weight of tricalcium phosphate with respect to the total weight of the food supplement is used, and more advantageously approximately 5% by weight.

Hydrated silica, magnesium stearate and colloidal silica may advantageously be used as thinners in the food supplement in the form of tablets or capsules. They are introduced in a quantity of approximately 2% by weight, 1% by weight and 0.6% by weight with respect to the total weight of the food supplement, respectively.

Other adjuvants such as flavourings (natural or chemical, fruit or other flavourings) or pigments are advantageously incorporated into the food supplement preparation.

When the food supplement is in the form of a soft capsule or a capsule, the envelope of these soft capsules or these capsules may in particular contain animal gelatine such as fish gelatine, glycerine, or a material of plant origin such as a cellulose or starch derivative, or a plant protein. In a preferred embodiment, one or more glycosylated bacterioruberins in accordance with the invention incorporated into the capsules may be dissolved in a fatty substance, advantageously caprylic and/or capric triglyceride, and preferably stabilized with tocopherol. Thus, a food grade of the MIRORUBERINE starting material marketed by GREENTECH and corresponding to the INCI names caprylic/capric triglyceride & tocopherol & Micrococcus lysate may be used in the food supplements in accordance with the invention.

The manner in which the invention may be implemented and the concomitant advantages will become more apparent from the following exemplary embodiments, which are given by way of non-limited indication and with the support of the accompanying figures.

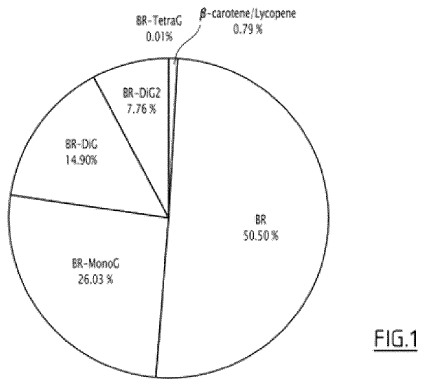

FIG. 1 shows the composition of an extract of carotenoids from the isolate SB5 of the species A. agilis.: BR=α-bacterioruberin, BR-MonoG: monoglycosylated form, BR-DiG: diglycosylated form, BR-DiG2: another form of BR-DiG, BR-TetraG: tetraglycosylated form.

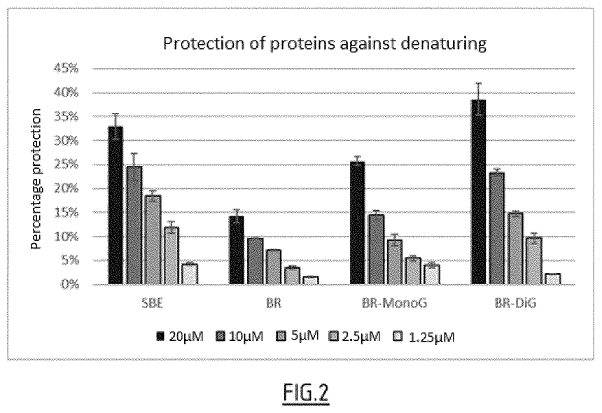

FIG. 2 shows the percentage protection against heat for a total carotenoid extract from A. agilis (Snow bacteria extract→SBE), bacterioruberin (BR), monoglycosylated bacterioruberins (BR-MonoG) and diglycosylated bacterioruberins (BR-DiG).

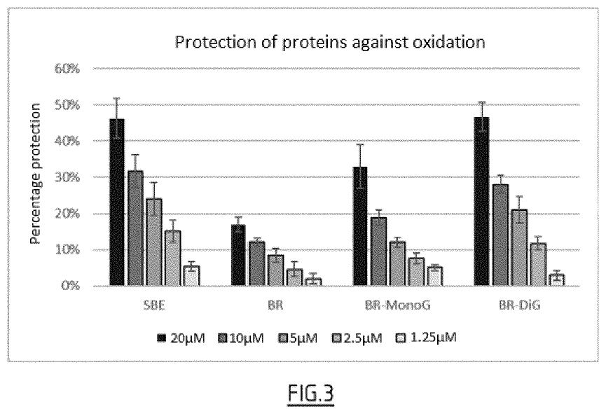

FIG. 3 shows the percentage of protection against oxidative stress for a total carotenoid extract from A. agilis (Snow bacteria extract→SBE), bacterioruberin (BR), monoglycosylated bacterioruberins (BR-MonoG) and diglycosylated bacterioruberins (BR-DiG).

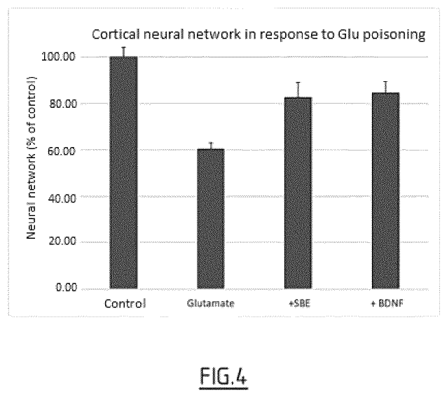

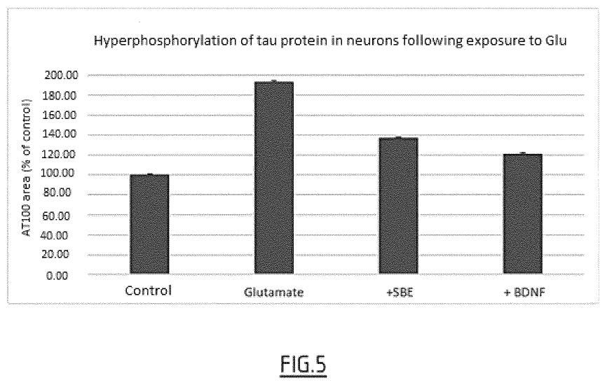

FIGS. 4 and 5 respectively represent the results for a neural network (FIG. 4) and tau hyperphosphorylation (FIG. 5) of cortical neurons damaged by glutamate and the protection conferred by the neurotrophin BDNF and the carotenoids of A. agilis (SBE). The results are expressed as a percentage of the control condition in the form of a mean+/−standard error (n=4-6). Statistical processing: One-way ANOVA followed by Fisher's Minimal Significant Difference (LSD) test. *=p<0.05 was considered significant.

### EXAMPLES

### Example I: Purification of Glycosylated Bacterioruberins from an Extract of Carotenoids of the Bacterium A. agilis

### I-1 Aim of the Study

The aim of this study was to isolate and quantify the molecules contained in an extract of total carotenoids from the bacterium A. agilis.

### I-2 Materials and Methods

### I-2.1 Extract

The total extract of carotenoids from the bacterium A. agilis contained in the GREENTECH starting material known as “Miroruberine” and corresponding to the INCI name Micrococcus lysate was used in this study; it was derived from the strain SB5. This so-called “SBE” extract could be obtained by using the method described in the publication of patent application WO 2014/167247.

### I-2.2 Column and Thin-Layer Chromatography

The SBE extract was taken up in tetrahydrofuran (THF) until it had completely dissolved. A step for separating the glycosylated bacterioruberins by silica gel chromatography was carried out in a glass column after diluting the SBE in THF.
1. Suspension of the silica gel in a DCM/methanol mixture (10/1) before being poured into the column
2. After sedimentation of the silica gel, 1 cm of sand is added before carrying out 3 washes with the DCM/methanol mixture.
3. 0.5 mL of SBE diluted in THF was deposited on the sand and allowed to stand for 5 minutes
4. 50 mL of DCM/methanol mixture (10/1) was added slowly, allowing fractions 1, 2, 3 and 4 to be collected separately
5. 40 mL of DCM/methanol mixture (8/2) was added slowly, allowing fractions 5 and 6 to be collected separately
6. 40 mL of DCM/methanol mixture (5/5) was added slowly, allowing fraction 7 to be collected
7. 40 mL of DCM/methanol mixture (3/7) was added slowly, to allow fraction 8 to be collected
8. All of the fractions were then compared by TLC (DCM/methanol (10/1)) with the SBE and quantified by absorption (using the absorption maximum for each fraction).

### I-2.3 Separation of Fractions by HPLC

Equipment: Nexera XR, binary pump (Shimadzu)
Column: C18; Intersustainable Swift 5 μm 4.6×150 mm, manufacturer: GL Sciences

### Mobile Phases:

A: 20% H₂O in MeOH
B 20% EtOAc in MeOH
Flow: 1.5 mL/min
Injection volume: 50 μL

TABLE 1

| | A | B |
|---|---|---|
| 1 min | 100 | 0 |
| 20 min | 0 | 100 |

### I-3 Results and Discussion

In this purification, the first step of separation by column chromatography made it possible to collect the various fractions the purity of which was confirmed by TLC and by HPLC-DAD, by comparing it with the absorbance spectrum of the native extract; the quantification of the various forms was carried out by UV absorption at 500 nm.

Each of the molecules of each of the fractions collected by chromatography was identified by Maldi-TOF-TOF spectroscopy using an AUTOFLEX instrument (Brucker). The method used was “CHCA and DHB Matrix without TFA in reflector acquisition”. The distribution between the different forms was calculated by combining the results obtained with the quantifications carried out on these fractions by HPLC-DAD (FIG. 1). Fraction 1 corresponds to beta-carotene, a by-product of the synthesis of bacterioruberin, but this molecule represented only 0.79% of the extract. Fraction 4 had the same profile for the Halobacter salinarium (Halorubin) extract, the majority molecule of which is bacterioruberin (BR). This molecule represented approximately half of the dry extract (FIG. 1).

Two diglycosylated forms (BR-DiG1 and BR-DiG2) which migrated separately were identified. The diglycosylated forms represented >22% of the extract.

The monoglycosylated form BR-MonoG represented >26% of the extract. The tetraglycosylated form (BR-TetraG) represented only 0.01% of the extract (FIG. 1).

### Example II: Protection and Stabilization of Proteins by an Extract of Carotenoids from A. agilis as Well as the Bacterioruberins and Glycosylated Forms Isolated Therefrom

### II-1 Aim of the Study

The aim of this study was to compare the protein protection capacities of the various components of an extract of carotenoids of the actinobacterium A. agilis separated by chromatography and HPLC. More specifically, we tested all the major fractions to assess their ability to protect the enzyme alkaline phosphatase:
against denaturing via their effect of protecting proteins from denaturing (AP-heat test) (chaperone effect)
against oxidation (APox test) (Protective effect against oxidative stress).

### II-2 Materials and Methods

### II-2.1 Test Samples

TABLE 2

| | |
|---|---|
| SBE | SBE = Snow bacteria extract; Total extract of |
| | carotenoids from A. agilis; this extract from the starting |
| | material corresponded to the INCI name i Micrococcus |
| | lysate (GREENTECH); extraction method described in |
| | patent application WO 2014-A-16727 |
| BR | α-bacterioruberin extracted from the |
| | GREENTECH starting material corresponding to the INCI |
| | name Micrococcus lysate (GREENTECH) isolated and |
| | purified in accordance with Example I |
| BR-MonoG | Monoglycosylated bacterioruberin extracted from |
| | the GREENTECH starting material corresponding to the |
| | INCI name Micrococcus lysate (GREENTECH) isolated |
| | and purified in accordance with Example I |
| BR-DiG | Diglycosylated bacterioruberin extracted from the |
| | GREENTECH starting material corresponding to the INCI |
| | name Micrococcus lysate (GREENTECH) isolated and |
| | purified in accordance with Example I |

Four doses were tested for SBE, BR, BR-MonoG and BR-DiG: 20 μM, 10 μM, 5 μM, 2.5 μM, 1.25 μM

### II-2.2 APox and AP-Heat Test

### APox Test and Protocol:

This test is described in patent application FR 3002544 A1 and measures the ability of a substance to protect the enzyme alkaline phosphatase from oxidative stress.

### Materials Needed:

Bovine alkaline phosphatase (AP) (Sigma P0114)
Liquid substrate for AP (Sigma P7998)
30% hydrogen peroxide
FeO₄S solution (30 mg in 1 mL H₂O)
Flat-bottomed 96-well plate
Plate reader at 405 nm
   The Following were Deposited in Each Well:
10 μL of AP diluted to 10⁻⁵ in 10⁻²M MgSO₄.
4 μL of test molecule, solvent (negative control) or H₂O (positive control)+6 μL of H₂O
30 μL of a hydrogen peroxide solution (from a stock solution composed of 940 μL H₂O+40 μL 30% H₂O₂+20 μL 10⁻² FeO₄S) or 30 μL of H₂O (positive control)
Allow to incubate for 15 min at 37° C.
Add 50 μL of liquid substrate.
Read OD at 405 nm for 20 min at 37° C.

### AP-Heat Test and Protocol:

This test is derived from a modification of the APox test for measuring the protection potential of proteins not just against oxidative stress, but against denaturing (heat stress). In fact, under the effect of heat, proteins are denatured and enzymes lose their activity. By adjusting the temperature and the incubation time, it is possible to determine the conditions necessary to inhibit 90% of the activity of the alkaline phosphatase (55° C. for 1 hour).

### Materials Required:

Bovine alkaline phosphatase (AP) (Sigma P0114)
Liquid substrate of alkaline phosphatase (Sigma P7998)
Heating block
Flat-bottomed 96-well plate
Plate reader at 405 nm

The following were deposited In each well:
10 μL of AP diluted to 10⁻⁵ in 10⁻²M MgSO₄
4 μL of test molecule or of solvent alone+6 μL of H₂O
Allow to incubate for 15 min, with stirring at 37° C.
Allow to incubate for 1 h at 37° C. or 55° C. on a heating block
Add 50 L of liquid substrate

Read OD at 405 nm for 20 min at 37° C.

The protective power of a “molecule X” against denaturing (PPX) is calculated by taking the ratio of the activity of alkaline phosphatase (PA) at 55° C. (stressed condition) to its activity at 37° C. (basal condition) in the presence of the molecule. This ratio is then normalized by means of the same ratio but obtained in the presence of the solvent alone.

Basically, the calculation was as follows:

$$PPX = ( {APA}_{X 55} - ( {APA}_{X 37} \times {APA}_{S 55} / {APA}_{S 37} ) ) / ( {APA}_{X 37} - ( {APA}_{X 37} \times {APA}_{S 55} / {APS}_{S 37} ) )$$

in which:
APA_{X55}: activity of AP in the presence of molecule X at 55° C.
APA_{X37}: activity of AP in the presence of the molecule X at 37° C.
APA_{S55}: activity of AP in the presence of the solvent at 55° C.
APA_{S37}: activity of AP in the presence of the solvent at 37° C.
and considering that:
APA_{X37} corresponds to 100% enzyme protection
APA_{X37}×APA_{S55}/APA_{S37} corresponds to zero protection.

The activity of the enzyme for each condition is calculated by taking the mean of the optical density values measured at 405 nm for the replicates, from which values the mean value of what are known as the “blank” wells (reagents alone) is subtracted, i.e.:

$$APA = [ ( OD replicate 1 - OD blank ) + ( OD replicate 2 - OD blank ) + ( OD replicate 3 - OD blank ) ] / 3$$

These calculations can only be applied with OD values located in the linear part of the curve, generally comprised between 0.15 and 1.5.

All the measurements for the activity of the enzyme were carried out on a 96-well EnSight-Perkin Elmer plate reader.

### II-3 Results and Discussion

The results of what is known as the “AP-heat” test are shown in FIG. 2. The glycosylated forms of the bacterioruberin (BR-MonoG, BR-DiG) as well as the SBE extract, which corresponds to a mixture of α-bacterioruberin (BR) and glycosylated forms of this carotenoid, in particular the diglycosylated form (BR-DiG), protect the protein more effectively than the BR itself. This effect was observed consistently for all of the test concentrations.

The results of the APOX test are shown in FIG. 3. These results show that the glycosylated forms have a stronger protection potential than the non-glycosylated BR and that this effect is consistent with a chaperone effect. In this case again, the protection of the oxidation protein is particularly pronounced for the diglycosylated form of bacterioruberin (BR-DiG) as well as for the total SBE extract. This effect was observed consistently for all of the test concentrations.

These results indicate that bacterioruberin (BR), but especially the glycosylated forms of bacterioruberin (in particular BR-MonoG, BR-DiG), as well as the SBE extract, which corresponds to a mixture of α-bacterioruberin (BR) and glycosylated forms of this carotenoid, and more particularly the diglycosylated form (BR-DiG), can be used to protect the intracellular proteins both from oxidative stress and from denaturing, which is generally followed by the formation of aggregates in the cells. Thus, bacterioruberin (BR), but especially the glycosylated forms of bacterioruberin (in particular BR-MonoG, BR-DiG), as well as the SBE extract, which corresponds to a mixture of α-bacterioruberin (BR) and glycosylated forms of this carotenoid, and more particularly the diglycosylated form (BR-DiG), are therefore suitable for their use for the development of treatment aimed at reducing the formation of toxic protein aggregates, for example in neurons, or in a disease involving a deregulation in protein aggregation.

### Example III: Neuroprotective Effect on a Neural Model

### III-1 Aim of the Study

The purpose of the study was to evaluate the neuroprotective effect of a carotenoid extract of the bacterium A. agilis, which is rich in “SBE” glycosylated bacterioruberins, on glutamate excitotoxicity in a cell model mimicking Alzheimer's disease (AD).

The glutamatergic system, and in particular the NMDA receptors (glutamatergic receptors) play a major role in learning processes and memory. Synaptic plasticity can be regulated by NMDA receptor signalling.

Overactivation of NMDA receptors is a common pathological feature in many neurodegenerative diseases, including those leading to cognitive impairment such as Alzheimer's disease. With this in mind, early pharmacological treatment with substances which reduce glutamate over-stimulation is a way to treat patients diagnosed with cognitive decline. tau is a microtubule-associated protein involved in microtubule stability and axonal transport. Pathological hyperphosphorylation of tau triggers the formation of neurofibrillary tangles and, in association with beta-amyloid protein oligomers, actively participates in the neurodegenerative process of AD. In addition, glutamate excitotoxicity and phosphorylation of the tau protein are closely related phenomena.

In this example, BDNF (Brain-Derived Neurotrophic Factor) was used as a positive control because this neurotrophin is known to promote survival and differentiation of neurons in vivo and in vitro.

### III-2 Materials and Methods

### III-2-1. Primary Culture of Cortical Neurons

All the experiments were carried out in accordance with the regulations in force in the European Union (Directive 2010/63/EU).

Murine cortical neurons were cultured as described by Callizot et al., 2013. The cells were mechanically dissociated by three forced passages through the tip of a 10 mL pipette. The cells were then centrifuged at 515×g for 10 minutes at 4° C. The supernatant was removed and the pellet was taken up in a defined culture medium constituted by Neurobasal medium with a 2% solution of B27 supplement, 2 mmol/litre of L-glutamine, 2% of PS solution and 10 ng/ml of BDNF. The viable cells were counted in a Neubauer cytometer, using the trypan blue exclusion test. The cells were seeded in a density of 25000 per well in a 96-well plate pre-coated with poly-L-lysine and were cultured at 37° C. in a CO₂ incubator (5%). The medium was changed every two days. The experiments were subsequently carried out on 96-well plates (n=6 culture wells per condition). In the 96 wells of each plate, only 60 were used. The wells in the first and last rows and columns were not used in order to avoid any edge effects and were filled with sterile water.

### III-2-2 Test Compounds and Glutamate Poisoning

The following compounds were tested in this example:

TABLE 3

| Test Compound | Concentration |
|---|---|
| Control (vehicle) | — |
| Glutamate | (20 μM, 20 min)/vehicle |
| SBE | 1 μm |
| BDNF | Homodimer 1.85 nM-50 ng/mL |

The compounds to be tested were dissolved in DMSO and the concentration was adjusted in order to ensure a concentration of DMSO in the culture medium of 0.1%. On day 13 of culture, the compounds were pre-incubated with primary cortical neurons for 1 hour, before the application of glutamate. Subsequently, on the same day, the cortical neurons were exposed to glutamate for 20 min. The glutamate was added at a final concentration of 20 μM (diluted in the control medium) in the presence of SBE or BDNF (used as a positive control). After 20 minutes, the glutamate was eliminated and fresh culture medium with the compounds under study was added for a further 48 hours.

### III-2-3 Evaluation of the Effects of the Compounds by Immunolabeling

48 hours after glutamate poisoning, the cell culture supernatant was removed using automatic multichannel pipettes. The cells were then washed with phosphate buffered saline (PBS). The cortical neurons were fixed with a cold solution of ethanol (95%) and acetic acid (5%) for 5 min at −20° C. They were washed twice again in PBS and then permeabilized. The non-specific sites were blocked with a PBS solution containing 0.1% saponin and 1% FCS, for 15 min at ambient temperature. The cells were incubated for 2 hours with respectively:
a) a mouse monoclonal anti-MAP-2 (microtubule associated protein 2) antibody at a dilution of 1/400 in PBS, with 1% foetal calf serum and 0.1% saponin. This antibody binds specifically to neurons and neurites, enabling the neural network to be studied.
b) a mouse monoclonal anti-phosphorylated tau AT100 antibody on Thr212/Ser214 at a dilution of 1/400 in PBS containing 1% foetal calf serum and 0.1% saponin. This antibody makes it possible to study the hyperphosphorylation of the tau protein.

These antibodies were revealed with the secondary antibodies Alexa Fluor 488 IgG goat anti-mouse, Alexa Fluor 568 IgG goat anti-chicken anti-mouse, Alexa Fluor 568 IgG goat anti-rabbit. These secondary antibodies were incubated with the neuron preparations at a dilution of 1/400 in PBS containing 1% FCS, 0.1% saponin, for 1 hour at ambient temperature.

For each condition, 30 images per well were automatically recorded using ImageXpress (Molecular Devices) at 20× magnification. All the images were generated using the same acquisition parameters. Analyses were automatically carried out from the images by Custom Module Editor® (Molecular Devices). The following parameters were examined:
total neural network (MAP-2 positive neural length)
hyperphosphorylation of tau protein (tau/MAP-2 overlap, μm² of overlap)

### III-2-4 Statistical Treatment of Data

The data were expressed as a percentage of the control. All of the values show the mean+/−standard error of the mean of 4-6 wells per condition. The graphs and statistical analyses for the various conditions (ANOVA followed by Fisher's LSD test [all groups versus glutamate group]) were carried out using GraphPad Prism software version 8.1.2. * P<0.05 was considered significant.

### III-3 Results and Discussion

Neural network integrity: Glutamate poisoning induced a significant reduction (60%) in neural network density (FIG. 4). As expected, BDNF exerts a significant protective effect on the integrity of the neural network (total neural network=85%,). The application of SBE considerably improved the integrity of the neural network. The total length of the neural network reached 83%, a significant result and comparable to that obtained with neurotrophin.

Hyperphosphorylation of the tau protein (AT100): Glutamate poisoning induced a significant increase in the AT100 zone corresponding to hyperphosphorylation of the tau protein and accumulation of the protein in the neuronal cytoplasm (+193% of the negative control (100%=value 0); FIG. 5). As expected, treatment with the neurotrophin BDNF resulted in a large and significant reduction in tau hyperphosphorylation (+121%). Like BDNF, the application of SBE significantly reduced the tau phosphorylation (+137%).

In summary, the SBE extract exerts a neuroprotective effect on the neural network and this effect is accompanied by a significant reduction in the hyperphosphorylation of the tau protein in the neuronal cytoplasm.

Thus, a bacterioruberin, preferably in the glycosylated form, optionally as a mixture with different forms of glycosylated bacterioruberins, or an extract comprising it, can be used in a method for treating or preventing a neurodegenerative disease.

### BIBLIOGRAPHY

Armstrong G A (1994) Eubacteria show their true colors-genetics of carotenoid pigment biosynthesis from microbes to plants. J Bacteriol. 176:4795-4802.
Armstrong, G A (1997). Genetics of eubacterial carotenoid biosynthesis: A colorful tale. In: Ornston, L N., editor. Annu Rev Microbiol. USA: Annual Reviews Inc.; pp. 629-659.
Britton G. (1995) Structure and properties of carotenoids in relation to function. FASEB J. 9:1551-1558.
Britton, GL-JSPH. (2004) Carotenoids Handbook. Basel; Boston: Birkhäuser Verlag. Callizot N, Combes M, Steinschneider R, Poindron P. (2013). Operational dissection of β-amyloid cytopathic effects on cultured neurons. J. Neurosci Res. 91:706-16.
Dugger B N, Dickson D W (2017) Pathology of Neurodegenerative Diseases Cold Spring Harb Perspect Biol 9 (7): a028035.
Farkhondeh T, Samarghandian S, Shaterzadeh Yazdi H, Samini F (2018) The protective effects of crocin in the management of neurodegenerative diseases: a review. Am J Neurodegener Dis 7:1-10.
Fong N, Burgess M, Barrow K, Glenn D (2001) Carotenoid accumulation in the psychrotrophic bacterium Arthrobacter agilis in response to thermal and salt stress Appl Microbiol Biotechnol 56, 750-756.
Mandelli F, Miranda V S, Rodrigues E, Mercadante A Z. (2012) Identification of carotenoids with high antioxidant capacity produced by extremophile microorganisms. world J Microbiol Biotechnol 28:1781-1790.
Strand A, Shivaji, S, Liaaen-Jensen (1997) Bacterial carotenoids 55. C50-carotenoids 25: revised structures of carotenoids associated with membranes in psychrotrophic Micrococcus roseus. Bioch. Syst. & Eco. 25 (6). 547-552

1-13. (canceled)

14. A method of treating or preventing a disease involving deregulation in protein aggregation in a subject, the method comprising:
administering to the subject a composition comprising at least one bacterioruberin,
wherein the disease is a neurodegenerative disease selected from Alzheimer's disease (AD), Parkinson's disease (PD), Huntington's disease, posterior cortical atrophy, and amyotrophic lateral sclerosis (ALS) or an ocular neurodegenerative disease selected from macular degeneration, retinitis pigmentosa, and retinopathy.

15. The method of claim 14, wherein the at least one bacterioruberin is a glycosylated bacterioruberin.

16. The method of claim 15, wherein the glycosylated bacterioruberin is a monoglycosylated bacterioruberin, diglycosylated bacterioruberin, triglycosylated bacterioruberin, tetraglycosylated bacterioruberin, pentaglycosylated bacterioruberin, hexaglycosylated bacterioruberin, heptaglycosylated bacterioruberin, octaglycosylated bacterioruberin, nonaglycosylated bacterioruberin, decaglycosylated bacterioruberin, undecaglycosylated bacterioruberin, or dodecaglycosylated bacterioruberin.

17. The method of claim 15, wherein the glycosylated bacterioruberin is monoglycosylated bacterioruberin, diglycosylated bacterioruberin, triglycosylated bacterioruberin, or tetraglycosylated bacterioruberins.

18. The method of claim 14, wherein the composition comprises a mixture of different forms of a glycosylated bacterioruberin.

19. The method of claim 14, wherein the neurodegenerative disease is AD or PD.

20. The method of claim 14, wherein the composition comprises at least one extract comprising at least one bacterioruberin and/or one glycosylated bacterioruberin.

21. The method of claim 14, wherein the composition comprises at least one bacterioruberin and one glycosylated bacterioruberin.

22. The method of claim 14, wherein the composition comprises a mixture of α-bacterioruberin, monoglycosylated bacterioruberin, and diglycosylated bacterioruberin.

23. The method of claim 14, wherein a ratio between the non-glycosylated forms and glycosylated forms of bacterioruberin is between 2/1 and 1/2.

24. The method of claim 14, wherein the composition is essentially free from non-glycosylated forms of bacterioruberin.

25. The method of claim 14, wherein the composition is formulated as a tablet, sugar-coated tablet, capsule, suppository, injectable or oral solution, or a drop.

26. The method of claim 14, wherein the composition is formulated for administration oromucosally, orally, rectally, vaginally, parenterally intramuscularly, or ophthalmically.

27. The method of claim 14, wherein the composition is in the form of a food supplement.

28. The method of claim 14, wherein the composition contains from 1 mg to 1 g of glycosylated bacterioruberins and/or non-glycosylated bacterioruberins or mixtures thereof.

29. A method of treating or preventing a disease involving a deregulation in protein aggregation in a subject, the method comprising:
administering to the subject a bacterioruberin,
wherein the disease is a neurodegenerative disease selected from Alzheimer's disease (AD), Parkinson's disease (PD), Huntington's disease, posterior cortical atrophy, and amyotrophic lateral sclerosis (ALS) or an ocular neurodegenerative disease selected from macular degeneration, retinitis pigmentosa, and retinopathy.

30. The method of claim 29, wherein the bacterioruberin is a glycosylated bacterioruberin.

31. The method of claim 29, wherein the bacterioruberin is a mixture of different forms of glycosylated bacterioruberins or an extract of bacterioruberin.

32. The method of claim 29, wherein the neurodegenerative disease is AD or PD.
